(19) United States
(12) Patent Application Publication
(10) Pub. No.: US 2012/0329985 A1
(43) Pub. Date: Dec. 27, 2012

(54) Optimal hydolysis conditions of soy protein to produce peptides with lipolysis-stimulating activity and their sequencing and use thereof

(75) Inventors: Wen-Dee Chiang, Taichung City (TW); Hao-Chun Kao, Taichung City (TW)

(73) Assignee: TUNGHAI UNIVERSITY, Taichung City (TW)

(21) Appl. No.: 13/337,136

(22) Filed: Dec. 25, 2011

(30) Foreign Application Priority Data
Jun. 21, 2011 (TW) 100121652

(51) Int. Cl. C12P 21/06; C07K 5/103

(52) U.S. Cl. 530/330; 435/68.1; 530/331

(57) ABSTRACT

This present invention discloses a method for preparing a lipolysis-stimulating soy protein hydrolysate, proceeding a hydrolysis reaction, which is a predetermined concentration of soy protein mediated by Flavourzyme in a predetermined hydrolysis conditions, wherein Flavourzyme versus the soy protein is 1:100, and the optimal hydrolysis conditions including reaction pH value 7˜7.5, reaction temperature 40˜50° C. and hydrolysis time 100˜150 minutes. This invention further discloses nine recombinations of isolated peptide sequences from the soy protein hydrolysate including Val-His-Val-Val, Leu-Leu-Leu, Leu-Leu-Ile, Leu-Ile-Leu, Leu-Ile-Ile, Ile-Leu-Leu, Ile-Leu-Ile, Ile-Ile-Leu and Ile-Ile-Ile.

### BACKGROUND OF THE INVENTION

According to the previous reports, the increased incidence of obesity is the trend in developed countries, especially, the incidence is positively correlated with consuming ability. The report from Department of Health, Executive Yuan in Taiwan also suggests that 25% of adults bear the problems in over-weight in 2009. According to the previous reports, imbalanced energy control is the major cause leading to over-weight and obesity. When the energy intake is more than energy expenditure in the organism, the excessive energy will be stored as triglyceride (TG) in the adipose tissue which is composed of adipocytes. More reports further indicate that the obesity is correlated with other diseases such as type-II diabetes, cardiovascular disorders, sleep apnea and cancers. The therapeutic approach for curing obesity could be achieved through stimulating lipolysis which degrades triglyceride in adipocytes and release glycerol from the cells. The lipolysis in adipose tissue has suggested as the good metabolic pathway which degrades the triglyceride into non-esterified fatty acid (NEFA) and glycerol.

The protein in food is one of the important nutritional origins to provide the required amino acid and energy for the maintenance of appropriated health and growth. Although the previous reports reveal that the animal proteins is more efficiently absorbed than vegetable proteins, some reports also suggest that vegetable protein is beneficial in decreasing blood lipid, cholesterol, and in retarding the progress of kidney disease caused by diabetes. Accompanied with increased understanding and knowledge, the protein hydrolysate originally provides for the patients with gastrointestinal damage is indicated to possess many bioactivities. For example, the protein hydrolysates contain the bioactivities in anti-oxidation, anti-bacteria, immunomodulation activity, decreasing high blood pressure, reducing cholesterol and triglyceride in the blood. In addition, these protein hydrolysates also possess the bioactivities for suppressing lipogenesis and stimulating lipolysis in 3T3-L1 adipocytes.

Recently, the usual methods used to generate the protein hydrolysate include acid hydrolysis, fermentation and enzymatic hydrolysis. The acid hydrolysis has the beneficial characteristics in low-cost, high hydrolysis efficiency and no bitter tast, however, the generation of carcinogens including monochloropropanol (MCP) and dichloropropanol (DCP) are accompanied with the hydrolysis. Furthermore, the neutralization during the acid hydrolysis will result in some bad effect such as high salt (more than 40% of sodium chloride) and high amount of monosodium glutamate (MSG) production. In fermentation method, Aspergillus oryzae used for protein hydrolysis is usually accompanied with production of volatile substances such as alcohol, organic acid, aldehydes and esters. Therefore, the fermentation method is usually used for generation of soy-bean sauce. In contrast to acid hydrolysis and fermentation, the enzymatic hydrolysis is simply accomplished by protease. Compared with fermentation method, enzymatic hydrolysis is more convenient to control the process. Compared with acid hydrolysis, the enzymatic hydrolysis does not produce the carcinogens such as MCP and DCP during the hydrolysis process. In addition, the enzymatic hydrolysis contains benefits including high reaction rate in normal pressure and low temperature, low energy cost and substrate specificity. Although the enzymatic hydrolysis possesses these advantages, the hydrolyzing efficiency and production rate of enzymatic hydrolysis is worse than acid hydrolysis. Fortunately, the hydrolyzing efficiency can be improved by modifying the hydrolysis conditions through changing the type of enzymes, pH value and ion concentration of hydrolyzing environment, reaction temperature and hydrolysis time. Moreover, the enzymatic hydrolysis will generate the hydrophobic peptides containing the bitter taste. Therefore, compounded enzymes such as Flavourzyme identified from Aspergillus oryzae by Novo Nordisk Company are used for enzymatic hydrolysis to avoid the bitter taste. Flavourzyme is a compounded protease, which possesses the enzyme activities as endopeptidase and exopeptidase, with advantages in high catalytic efficiency and less bitter taste.

Soybean is the food origin containing rich proteins (35%), lipid and other nutritions. After defatting, removing seed-coat and grinding into powder, the protein composition in defatted soy flour would reach to 50%. Following the treatment of acid and ethanol to remove saccharide and flavo compounds, the soy protein concentrate contains protein composition for 65˜70%. Furthermore, the protein composition of the soy protein concentrate is further enriched up to 85˜90% through treatment of alkaline solution, and is followed by centrifugation for removing soybean fiber. Finally, the isolated soy protein (ISP) is generated by protein precipitation through adding acid to reach the isoelectric point. According to the previous reports, either isolated soy protein or soy protein concentrate are sufficient for our requirement. Therefore, soy protein is the pure vegetable protein which able to replace the animal protein as the protein origin for human beings. In addition, the soy protein contains several bioactivities such as reducing the cholesterol and triglyceride in blood, suppressing appetite and reducing the blood pressure in hypertension patients. Furthermore, the advanced studies suggest that the soy protein hydrorlyzed by different enzyme generates different peptides containing better physiology activities than soy protein. For example, the ISP hydrolysate generated by Alcalase contains the peptide which is capable to suppress hypertension. The soy protein hydrolyzed by microorganism could retard the oxidation of lipid in the meat. The ISP hydrolysate generated by the protease in Bacillus subtilis could significantly reduce the blood lipid and body fat content in the rat.

Besides the bioactive peptides above, many studies also aim for investigating the ISP hydrolysate prepared by Flavourzyme and Neutrase, because these hydrolysates possess the bioactivity for stimulating lipolysis in 3T3-L1 adipocytes. In the future, these hydrolysates prepared by Flavourzyme and Neutrase could be applied in obesity therapy. The hydrolysis efficiency is determined by many factors, therefore, it is necessary to identify the most appropriated hydrolyzing environment and hydrolysis time to improve the hydrolyzing efficiency and bioactivities of hydrolysates. Especially, Flavourzyme is a kind of compounded protease which possesses the difficulty in indetifying the most appropriated hydrolysis condition. The investigators had tried to determine the enzyme activity of Flavourzyme for hydrolyzing 8% ISP by using the experimental design in “one factor at a time” model to identify the most appropriated reaction condition. However, the result obtained from this experimental design neither reveals the interaction between investigated factor and other factors, nor describes the most appropriated reaction environment. Therefore, it is necessary to modify the experimental design for determining the interaction between different effecting factors; otherwise, we have to expand the experiment scale which results in the waste in time and cost. Moreover, increased experiment number without any modification in the experimental design would not identify the most appropriated condition due to the obvious interaction between different variables.

### SUMMARY OF THE INVENTION

Base the foregoing, one aspect of this invention is to provide a method for preparing a lipolysis-stimulating soy protein hydrolysate, proceeding a hydrolysis reaction with an optimal hydrolysis condition for obtaining a soy protein hydrolysate with best bioactivity of lipolysis-stimulating, wherein:

The method with a predetermined concentration of soy protein is mediated by Flavourzyme in the optimal hydrolysis condition including pH value 7˜7.5, reaction temperature 40˜50° C. for 100˜150 minutes.

The soy protein includes defatted soy flour, soy protein concentrate, isolated soy protein (ISP) and other processed soy protein, of which the best one is the isolated soy protein, and a best ratio of the soy protein and Flavourzyme is 100:1.

Furthermore, the soy protein hydrolysate obtained by the method has the bioactivity for stimulating lipolysis.

Another aspect of the invention is to provide an isolated functional peptide having a amino acid sequence shown below in (1) or (2):

(1) Val-His-Val-Val.

(2) the peptide is composed of three amino acids, wherein:

the first amino acid is Leu or Ile,

the second amino acid is Leu or Ile, and

the third amino acid is Leu or Ile.

And the isolated functional peptide consequentially obtained by hydrolyzing a soy protein with Flavourzyme has the bioactivity for lipolysis to increase the glycerol release in adipocytes of an organism, wherein, the soy protein is selected from defatted soy flour, soy protein concentrate, ISP or other processed soy protein, and the best one is ISP.

In another aspect of the invention is also to provide a medical compound for reducing weight, wherein an efficient component is a isolated peptides obtained from a soy protein hydrolysate having an amino acid sequence as below (1) or (2):

(1) Val-His-Val-Val.

(2) the peptide is composed of three amino acids, wherein:

the first amino acid is Leu or Ile,

the second amino acid is Leu or Ile, and

the third amino acid is Leu or Ile.

The present invention can be best understood through the following description and accompanying drawings, wherein:

### BRIEF DESCRIPTION OF THE DRAWINGS

FIG. 1 is the effect of reaction temperature and pH in 120 min hydrolysis time (HT) on glycerol release in 3T3-L1 adipocytes.

FIG. 2 is the effect of hydrolysis time and pH value at 50° C. reaction temperature on glycerol release in 3T3-L1 adipocytes.

FIG. 3 is the Effect of hydrolysis time (HT) and reaction temperature (RT) at reaction pH 7 on glycerol release in 3T3-L1 adipocytes.

FIG. 4 is the molecular weight distribution for retentates and permeate obtained from the fractionating ISP hydrolysate with different molecular weight cut-off ultrafiltration membranes.

FIG. 5 is the bar graph to present the effect ISP hydrolysate and its membrance fractions on glycerol release in 3T3-L1 adipocytes.

FIG. 6 is the bar graph to present the effect ISP hydrolysate and its membrance fractions on triglyceride residue in 3T3-L1 adipocytes.

FIG. 7 is the quantitated bar graph to present the expression of HSL in 3T3-L1 adipocytes after treatment with 1 kDa retentate fraction within different cultured time.

FIG. 8 is the quantitated bar graph to present the expression of phosphorylated HSL in 3T3-L1 adipocytes after treatment with 1 kDa retentate fraction within different cultured time.

FIG. 9 is the gel filtration spectrum chromatography of ISP hydrolysate 1 kDa retentate ISP hydrolysate obtained from ultrafiltration.

FIG. 10 is the bar graph to show the effect of ISP hydrolysate 1 kDa retentate fraction and its gel filtration fractions on glycerol release in 3T3-L1 adipocytes.

FIG. 11 is the bar graph to show the effect of ISP hydrolysate 1 kDa retentate fraction and its gel filtration fractions on triglyceride residue in 3T3-L1 adipocytes.

FIG. 12 is the bar graph to show the dosage effect of GF3 fraction on glycerol release in 3T3-L1 adipocytes.

FIG. 13 is the bar graph to show the dosage effect of GF3 fraction on triglyceride residue in 3T3-L1 adipocytes.

FIG. 14 is the high-performance liquid chromatography of GF3 fraction.

FIG. 15 is the bar graph to show the effect of GF3 fraction and its reverse phase chromatography fractions on glycerol release in 3T3-L1 adipocytes.

FIG. 16 is the bar graph to show the effect of GF3 fraction and its reverse phase chromatography fractions on triglyceride residue in 3T3-L1 adipocytes.

FIG. 17 is the high-performance liquid chromatography of HF4 fraction.

FIG. 18 is the bar graph to show the effect of HF4 fraction and its reverse phase chromatography fractions on glycerol release in 3T3-L1 adipocytes.

FIG. 19 is the bar graph to show the effect of HF4 fraction nd its reverse phase chromatography fractions on triglyceride residue in 3T3-L1 adipocytes.

FIG. 20 is the mass spectrum of RHF4-2.

FIG. 212 is the mass spectrum of RHF4-3.

FIG. 22 is the bar graph to show the effect of RHF4-2, RHF4-3 fractions and the synthetical peptides on glycerol release in 3T3-L1 adipocytes.

FIG. 23 is the bar graph to show the effect of RHF4-2, RHF4-3 fractions and the synthetical peptides on triglyceride residue in 3T3-L1 adipocytes.

FIG. 24 is the bar graph to show the effect of Leu-Leu-Leu following pre-incubation with gastrointestinal protease on glycerol released in 3T3-L1 adipocytes.

FIG. 25 is the bar graph to show the effect of Val-His-Val-Val following pre-incubation with gastrointestinal protease on glycerol released in 3T3-L1 adipocytes.

FIG. 26 is the bar graph to show the effect of Leu-Leu-Leu following pre-incubation with gastrointestinal protease on triglyceride residue in 3T3-L1 adipocytes.

FIG. 27 is the bar graph to show the effect of Val-His-Val-Val following pre-incubation with gastrointestinal protease on triglyceride residue in 3T3-L1 adipocytes.

FIG. 28 is the bar graph to show the effect of Leu-Leu-Leu and Val-His-Val-Val on glycerol released in 3T3-L1 adipocytes in the presence of insulin.

FIG. 29 is the bar graph to show the effect of Leu-Leu-Leu and Val-His-Val-Val on triglyceride residue in 3T3-L1 adipocytes in the presence of insulin.

### DETAILED DESCRIPTION OF THE PREFERRED EMBODIMENTS

This invention discloses a method of preparing a soy protein hydrolysate. First, a prepared ISP at a predetermined concentration is treated with Flavourzyme, then proceeding a hydrolysis reaction in the optimal hydrolysis conditions to obtain a ISP hydrolysate with best bioactivity for lipolysis, wherein the ISP versus Flavourzyme is 100:1, and the optimal hydrolysis condition is at pH value 7.0˜7.5, reaction temperature 40˜50° C. and hydrolysis time 100˜150 minutes. This invention further identifies nine combinations of amino acid sequences from the ISP hydrolysate, including Val-His-Val-Val [symbol] Leu-Leu-Leu [symbol] Leu-Leu-Ile [symbol] Leu-Ile-Leu [symbol] Leu-Ile-Ile [symbol] Ile-Leu-Leu [symbol] Ile-Leu-Ile [symbol] Ile-Ile-Leu and Ile-Ile-Ile.

In order to clearly describe this invention in the following, the examples with tables and figures are used to examine this invention.

It is necessary to emphasize that hydrolysis of soy protein is affected by multiple factors which interacts to each other. Therefore, the hydrolysis is observed according to method of steepest ascent, which utilizes central composite design to mimic the reaction state of the starting point by asterisms, pivot points and central points. The experimental results are further analyzed by response surface methodology (RSM), which combines mathematics and statistics for analyzing the effect of each variable, to identify the optimal reaction conditions for the hydrolysis.

Furthermore, both lipogenesis and lipolysis occur in the adipocyte, wherein the lipolysis means the hydrolyzing process of triglyceride mediated by three lipases including adipose triglyceride lipase (ATGL), hormone-sensitive lipase (HSL) and monoglyceride lipase to produce free fatty acid and glycerol. The produced glycerol will be released to extracellular space of the adipocyte due to its poor availability for cells. Therefore, the glycerol released from the adipocyte or the triglyceride retained in the adipocyte could be utilized as the parameter for the determining the lipolysis efficiency.

Therefore, measurement of the glycerol released from 3T3-L1 adipocyte is used for determining the optimal hydrolysis conditions.

### Example 1

### Preparation of Isolated Soy Protein (ISP) Hydrolysates

The commercialized Flavourzyme® Type A is purchased from Novo Industry A/S (Copenhagenm Denmark), and the ISP is purchased from Chen-Fang company (Taiwan).

First, Flavourzyme is added into 2.5% ISP with the substrate-enzyme ratio at 100:1. According to the previous reports, three factors affecting protein hydrolysis are pH value, hydrolysis time (HT, minutes) and reaction temperature (RT, ° C.). Therefore, we use central composite design which contains three variables and five levels to obtain the hydrolysis parameter listed in table 1.

TABLE 1
Hydrolysis parameters in central composite design (Three variable and
five levels)

| Independent | Code level of variable | | | | |
|---|---|---|---|---|---|
| variable | −1.68 | −1 | 0 | 1 | 1.68 |
| PH (X1) | 5.32 | 6 | 7 | 8 | 8.68 |
| Reaction | 33.2 | 40 | 50 | 60 | 66.8 |
| temperature | | | | | |
| (X2) (° C.) | | | | | |
| Hydrolysis time | 19.2 | 60 | 120 | 180 | 220.8 |
| (X3) (min) | | | | | |

Therefore, the mixed solutions are reacted for the hydrolysis with different conditions described in table 1. After finish of the hydrolysis time, the hydrolysate is incubated in the boiling water for 15 minutes to terminate the enzyme activity and followed by cooling. The supernatant is collected after centrifugation at 9000×g for 15 minutes for freeze-drying to obtain the ISP hydrolysate (ISPH) for the following experiments.

### Example 2

### Culture of 3T3-L1 Adipocytes

The precursor cells of 3T3-L1 cell are purchased from Food Industry Research and Development Institute in Taiwan. The purchased precursor cells of 3T3-L1 adipocytes are cultured in 24-wells plate with 1×10⁴ cells/well. The cells are cultured with DMEM (Dulbecco's Modified Eagle Medium) containing 10% FBS (Fetal bovine serum) at 37° C. in the incubator with 5% CO₂, and refresh cultured medium every two days. While the 3T3-L1 cells are filled within the culture dish, the culture medium is changed to the differentiation medium (DM) for promoting adipocytes differentiation, which is defined as day 0 post-differentiation. The differentiation medium contains 1.74 μM insulin, 0.86 mM dexamethasone (DEX) and 0.5 mM isobutyl-methylxanthine (IBMX). From day 2 post-differentiation, the culture medium is exchanged to DMEM with 1.74 μM insulin and refreshed every two days until day 8 post-differentiation. On day 8 post-differentiation, the precursor cells would differentiate into mature 3T3-L1 adipocytes.

### Example 3

### Measurement of the Glycerol Released from 3T3-L1 Adipocytes

The 3T3-L1 adipocytes cultured in example 2 are washed by PBS (phosphate buffered saline), and respectively added with 400 ppm ISP hydrolysates prepared according to different hydrolysis condition in example 1 for the following culture until day 11 post-differentiation.

30 μL of the cultured medium is collected and mixed with detecting kit (GY105) to measure the glycerol release. After reaction at room-temperature for 5 minutes, the absorption excited by 520 nm wavelength is measured by spectrophotometer to calculate the glycerol released from adipocytes. The results are showed in table 2:

TABLE 2
Glycerol released from 3T3-L1 adipocytes

| | Code level of each variable | | | |
|---|---|---|---|---|
| | PH | RT | HT | Released glycerol |
| Number | (X1) | (X2) (° C.) | (X3) (min) | (nmol/mg protein) |
| 1 | −1 | −1 | −1 | 352.26 |
| 2 | −1 | −1 | 1 | 340.56 |
| 3 | −1 | 1 | −1 | 344.06 |
| 4 | −1 | 1 | 1 | 339.27 |
| 5 | 1 | −1 | −1 | 345.20 |
| 6 | 1 | −1 | 1 | 352.79 |
| 7 | 1 | 1 | −1 | 344.86 |
| 8 | 1 | 1 | 1 | 351.15 |
| 9 | 0 | 0 | −1.68 | 346.98 |
| 10 | 0 | 0 | 1.68 | 350.52 |
| 11 | 0 | −1.68 | 0 | 349.60 |
| 12 | 0 | 1.68 | 0 | 346.99 |
| 13 | −1.68 | 0 | 0 | 344.09 |
| 14 | 1.68 | 0 | 0 | 343.23 |
| 15 | 0 | 0 | 0 | 362.68 |
| 16 | 0 | 0 | 0 | 358.55 |
| 17 | 0 | 0 | 0 | 358.14 |
| 18 | 0 | 0 | 0 | 361.93 |
| 19 | 0 | 0 | 0 | 357.59 |

After analysis of the results in table 2 by central composite design, the amounts of glycerol released from 3T3-L1 adipocytes are 339.27352.79 nmol/mg protein.

### Example 4

### Analyzing of the Optimal Hydrolysis Condition

The 19 results in table 2 are analyzed by the RSREG, which is a statistical analysis system, to obtain a second-order model equations shown below:

Y=55.43+68.35X₁+3.12X₂−0.24X₃−5.67X₁²−0.04X₂²−0.0011X₃²+0.094X₁X₂+0.0634X₁X₃+0.0012X₂X₃

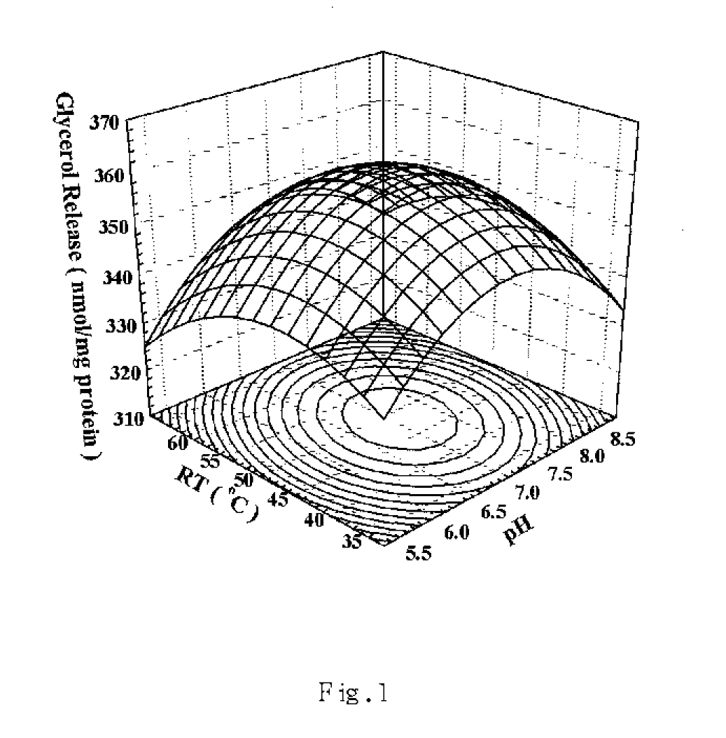

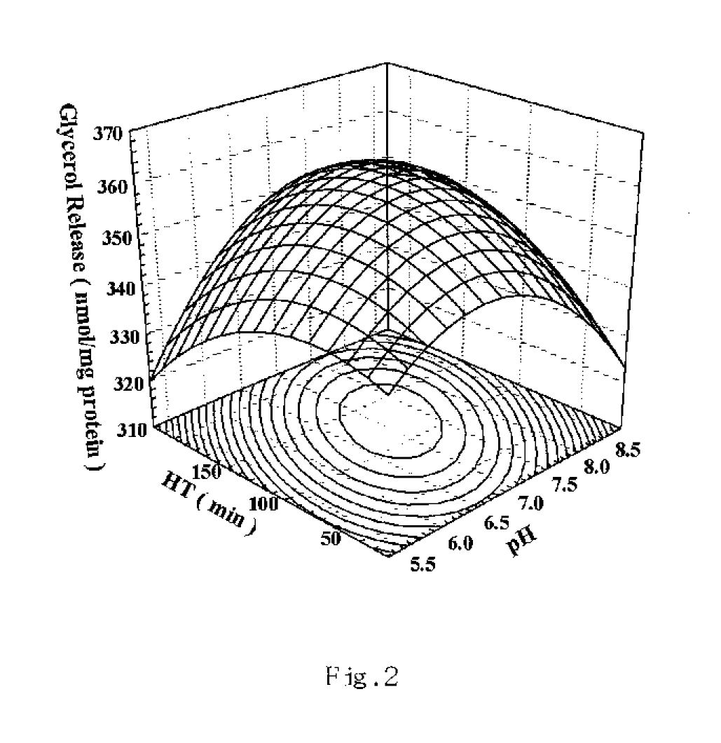

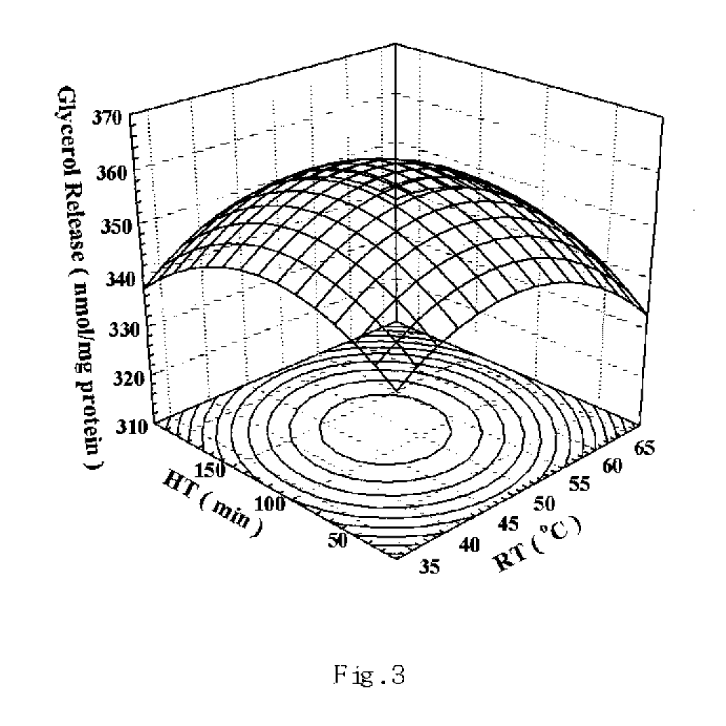

According to this polynomial model, we can draw the surface figure according the glycerol release with two variables when we fix another variable, and show the results in FIG. 1 to FIG. 3. In FIG. 1, we fix the hydrolysis time at 120 minutes for detecting the effect of pH value and reaction temperature on glycerol released from 3T3-L1 adipocytes. In FIG. 2, we fix the reaction temperature at 50° C. for detecting the effect of pH value and hydrolysis time on glycerol released from 3T3-L1 adipocytes. In FIG. 3, we detect the effect of hydrolysis time and reaction temperature on glycerol released from 3T3-L1 adipocytes when environmental pH value is 7.0.

Taken together, the most amount of glycerol released from 3T3-L1 adipocytes is achieved when the ISP is hydrolyzed at 40˜50° C., pH value 7˜7.5 for 100˜150 minutes according to the result in FIG. 1 to FIG. 3. The condition estimated by the quadratic polynomial model also shows that the optimal hydrolysis condition for obtaining the most glycerol released from 3T3-L1 adipocytes is conducted at 48.8° C., pH value 7.12 for 124.9 minutes.

The optimal estimated lipolysis condition mediated by ISP hydrolysate is further confirmed by six independent assays, and the results are showed in table 3:

TABLE 3
Confirmation of the effect in glycerol release promoted by ISP
hydrolysate prepared by the estimated hydrolysis conditions

| Response variables | Glycerol release (nmol/mg protein) |
|---|---|
| Predict value | 359.93 |
| Experimental value (mean) | 359.92 ± 18.53 |
| Sample Size | 6 |
| 95% Confidence interval | (340.48, 379.36) |

Table 3 reveals that the observation of glycerol released from 3T3-L1 adipocytes is 359.92 nmol/mg protein and the prediction is also in 95% confidence interval. Therefore, the hydrolysis progressed under this optimal hydrolysis condition can acquire the ISP hydrolysate to produce the most amount of glycerol which is suggested as the most efficient lipolysis.

### Example 5

### Separation of the ISP Hydrolytes by Molecular Weight Cut-Off Ultrafiltration Membrane

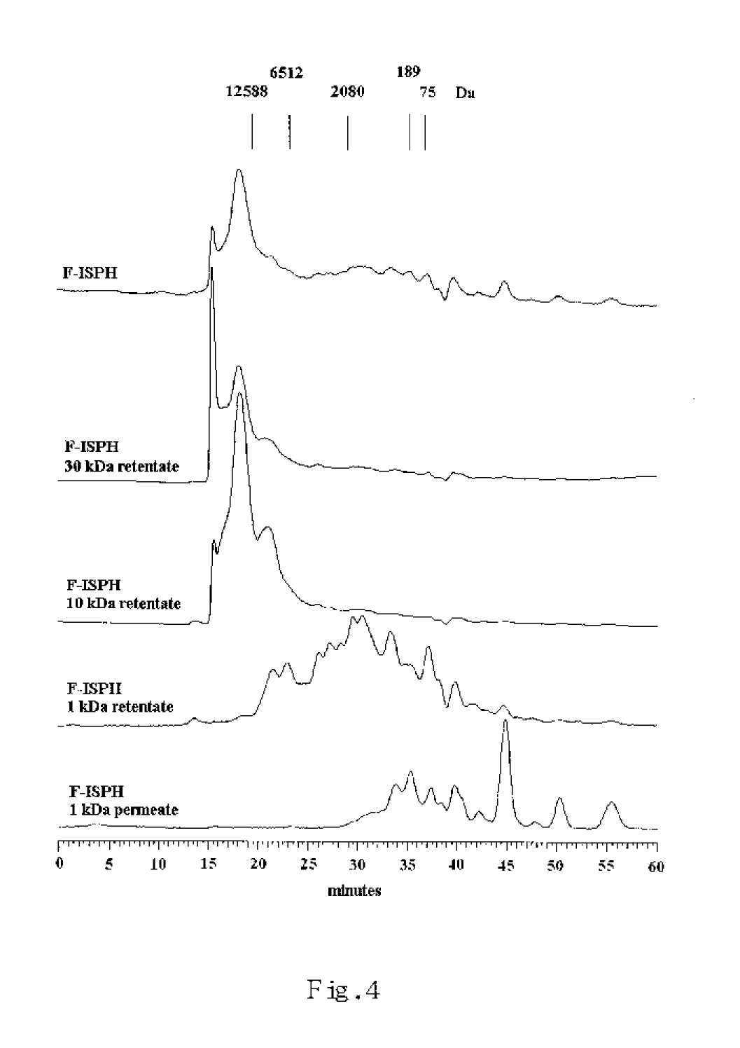

In this example, the supernatant of ISP hydrolysate is collected after centrifugation, and separated by different molecular weight cut-off (MWCO) ultrafiltration membranes including 30 kDa, 10 kDa and 1 kDa MWCO ultrafiltration membranes. The fractions with the molecular weight of 30 kDa retentate, 10 kDa retentate, 1 kDa retentate and 1 kDa permeate are collected for the freeze-drying. The powder of each fractions are dissolved at same concentration for the analysis of HPLC (High performance liquid chromatography) and gel chromatography to determine the spectrum of molecular weight in each fraction which is showed in FIG. 4.

The results show that the molecular weight of the majority of ISP hydrolysate is more than 12588 Da when compared to the standards with different molecular weight including 12588, 6512, 2126, 189 and 75 Da. The molecular weight of content in 30 kDa retentate of ISP hydrolysate is mainly more than 12588 Da, and the molecular weight of major content in 10 kDa retentate is 12588 Da and minor content is between 2126-12588 Da. In addition, the molecular weight of the major content in 1 kDa retentate is between 2126-12588 Da and minor content is less than 2126 Da. Finally, spectrum of molecular weight of the content in 1 kDa permeate is mainly less than 2126 Da.

### Example 6

### Measurement of the Glycerol Release Promoted by Different Fractions

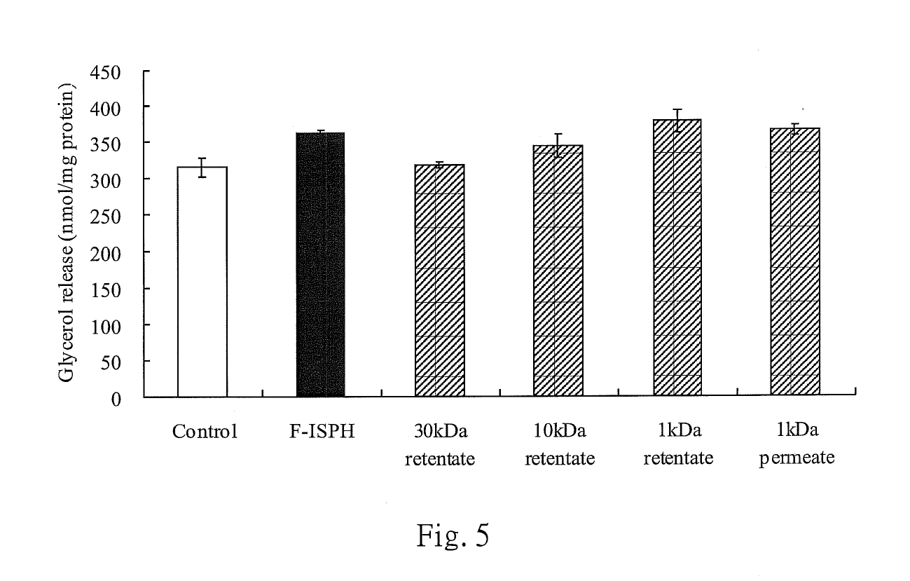

400 ppm ISP hydrolysate and each fraction prepared in example 5 are used to determine the effects of the fractions on glycerol release in 3T3-L1 adipocytes. The results showed in FIG. 5 which contains the control experiment without treatment of ISP hydrolysate or fractions.

The results in FIG. 5 reveal that not only the ISP hydrolysate promote the glycerol release to 363.13 nmol/mg protein, but also 10 kDa retentate, 1 kDa retentate and 1 kDa permeate show the obvious promotion in the glycerol release. However, the glycerol release detected in control cells or the cells treated with 30 kDa retentate do not show the obvious difference. Among all assays, the 3T3-L1 cells treated with 1 kDa retentate shows the most glycerol release which is up to 378.19 nmol/mg protein in 3T3-L1 adipocytes.

### Example 7

### Measurement of the Triglyceride Residue in 3T3-L1 Adipocyte Treated Different Fractions

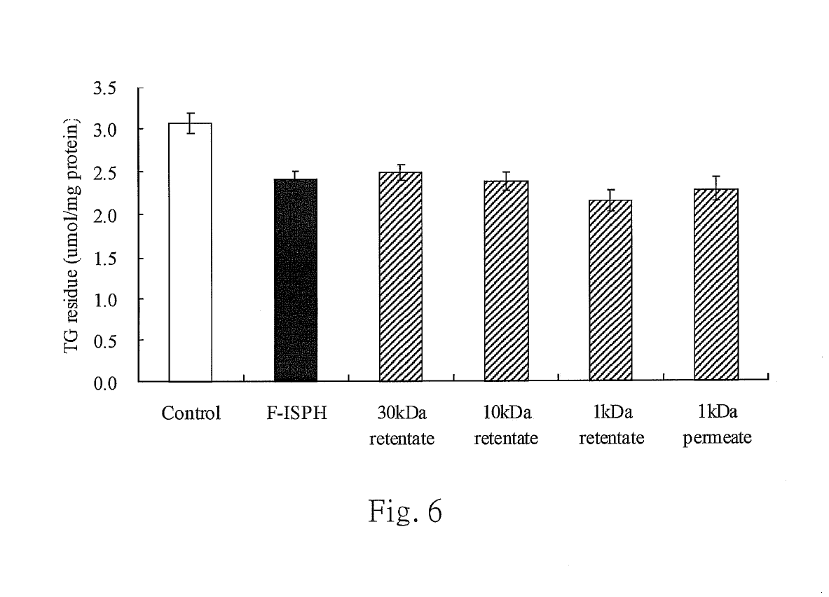

The 3T3-L1 cells cultured in example 2 are washed by PBS and further respectively cultured with medium containing 400 ppm ISP hydrolysate or different fractions prepared in example 5 until day 11 post-differentiation. On day 11, the cultured cells are washed with PBS and lysed by adding lysis buffer. The supernatant of cell lysis is collected after centrifugation at 13000 rpm for 10 minutes. 10 μL supernatant of cell lysis is mixed with 1 ml triglyceride detecting kit (TR213) and incubated at room-temperature for 5 minutes. After the incubation, the triglyceride residue in the adipocytes could be calculated according to absorption excited by 500 nm wavelength and shown in FIG. 6. In FIG. 6, control experiment is the cells without treatment of ISP hydrolysate or any fractions.

The results in FIG. 6 suggest that the triglyceride residue in the 3T3-L1 adipocytes cultured with 400 ppm ISP hydrolysate is 2.42 μmol/mg protein which is significantly less than 3.08 μmol/mg protein in the control cells. In addition, the cells treated with different fractions also shows the reduction of triglyceride residue in 3T3-L1 adipocytes, therein the 3T3-L1 cells treated with 1 kDa retentate revealed the least triglyceride residue at 2.16 μmol/mg protein which is also significantly less than the residue in cells cultured with ISP hydrolysate.

According to the results in example 6 and example 7, culture adipocyte with treatment of 1 kDa retentate would increase the glycerol release from 15% to 20% and reduce the triglyceride residue from 21% to 30% in the cells when compared with the adipocytes treated with ISP hydrolysate. Therefore, these data suggest that the composition in 1 kDa r retentate contains the best bioactivity for lipolysis.

### Example 8

### Preparation of the 1 kDa Retentate from ISP Hydrolysate

According to the preparing method in example 1, the 2.5% ISP is hydrolyzed by Flavourzyme at pH value 7.0, reaction temperature 50° C. for 2 hours to produce ISP hydrolysate.

According to the flow chart in example 5, the ISP hydrolysate are subsequently separated by 30 kDa, 10 kDa, 1 kDa molecular weight cut-off ultrafiltration membranes to isolate the 1 kDa retentate for the further experiment in following examples.

### Example 9

### Expression Pattern of Hormone-Sensitive Lipase (HSL)

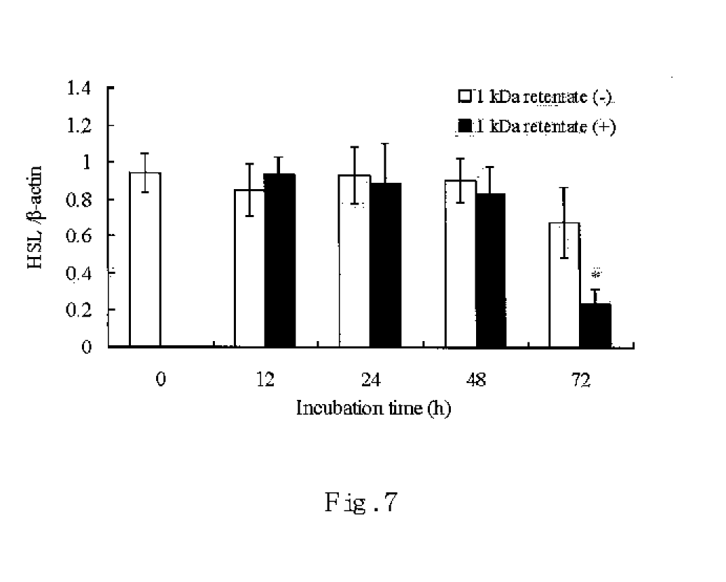

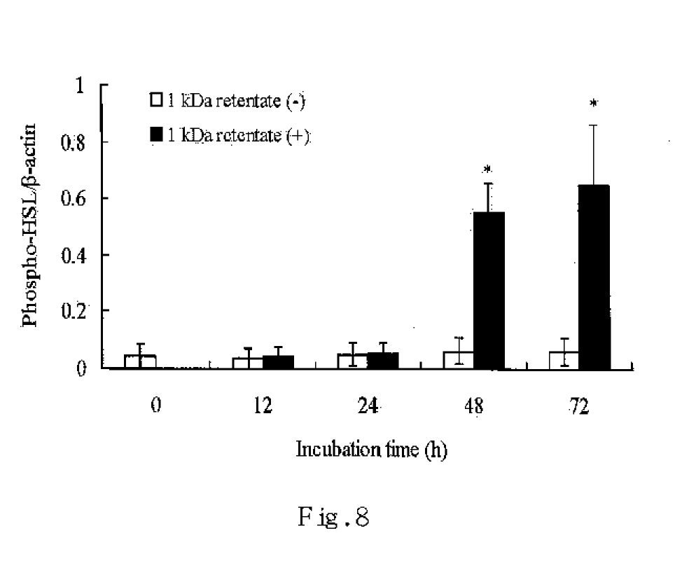

The 3T3-L1 adipocytes cultured in example 2 are further respectively cultured with medium containing 50 ppm 1 kDa retentate preparing in example 8 for 12, 24, 48 and 72 hours. After culture with different harvest time, the 3T3-L1 adipocytes are washed by PBS twice and lysed by lysis buffer. 10 μg extraction of the cell lysis mixed with sampling buffer is heated at 95° C. and followed by separation using electrophoresis with 10% SDS-PAGE. After transfer on the PVDF membrane, anti-HSL and anti-phosph HSL first antibodies are applied in the western blot and followed by staining with secondary antibody to characterize the expression pattern of HSL or phosphorylated HSL. The results are showed in FIGS. 7 and 8 and appendixes I and II, wherein the appendixes I is the gel electrophoresis to present the expression of HSL in 3T3-L1 adipocytes after treatment with 1 kDa retentate fraction within different cultured time, and the appendixes II is the gel electrophoresis to present the expression of phosphorylated HSL in 3T3-L1 adipocytes after treatment with 1 kDa retentate fraction within different cultured time.

In FIG. 7 and appendix I, after culture with 1 kDa retentate isolated from ISP hydrolysate for 24, 48 and 72 hours, the expression of HSL in the adipocytes is gradually reduced, therein the HSL expression in adipocytes is significantly reduced after culture for 72 hours.

In FIG. 8 and appendixes II, the expression of phosphorylated HSL in the adipocytes cultured with 1 kDa retentate isolated from ISP hydrolysate is significantly increased after culture for 48 and 72 hours.

Taken together, the ISP hydrolysate 1 kDa retentate is capable to reduce the HSL expression but promote the phosphorylation of HSL for activating lipase activity in the adipocytes after culture for 48 or 72 hours.

### Example 10

### Translocation of HSL by Immunostaining

The results in example 9 reveal that the phosphorylation of HSL is increased after culture for 48 or 72 hours with the ISP hydrolysate 1 kDa retentate. Therefore, the subcellular localization of HSL is further investigated by immunostaining. After culture for 48 or 72 hours, the adipocytes with different harvest time are fixed by PBS contains 4% formalin and 0.01% Triton X-100 at room-temperature for 20 minutes, and followed by PBS wash for three times.

The fixed adipocytes are covered by cold formaldehyde, which contains 5% fetal bovine serum, for incubation in the ice box to block the non-specific binding, and followed by PBS wash for three times. Furthermore, the cells are incubated with rabbit anti-phosphoryated HSL antibody at 4 over-night which is followed by the staining with FITC-conjugated donkey anti-rabbit IgG secondary antibody at room-temperature. The expression of phosphorylated HSL in the adipocytes is observed by microscope and shown in appendix III.

According to the immunostaining, the phosphorylated HSL is obviously congregated to peripheral zone of lipid droplet in adipocytes with treatment of 1 kDa retentate of the ISP hydrolysate for 48 and 72 hours when compared with control. As bright circles shown in two left panels in appendix III, the signal of immunostaining is obviously stronger in the peripheral zone of lipid droplet. Therefore, the data suggest that the treatment of 1 kDa retentate of ISP hydrolysate will trigger the translocation of phosphorylated HSL to lipid droplet for lipolysis.

### Example 11

### Separation of ISP Hydrolysate 1 kDa Retentate by Gel Chromatography

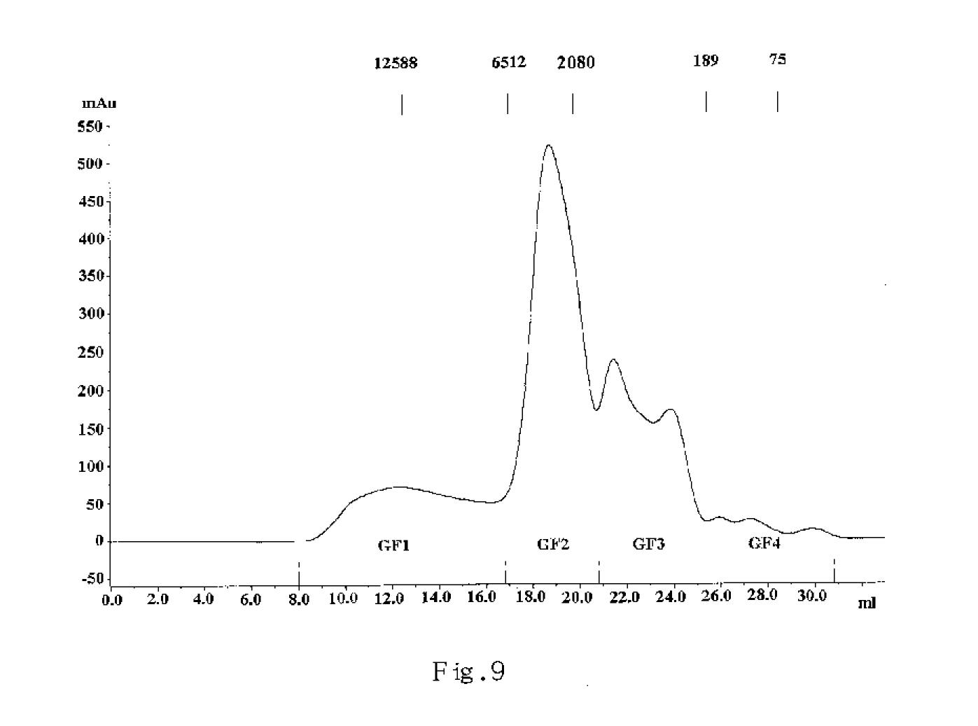

The mobile phase contains 30% acetonitrile and the ISP hydrolysate 1 kDa retentate at a predetermined concentration which is infiltrated with 0.22 μm filter. 500 μL of the mobile phase solution is injected into the column with the rate at 0.5 mL/minute to separate the molecules according to different polarity, and is detected with the absorption excited by 280 nm wavelength. As the result shown in FIG. 9, there are four fractions separated from the ISP hydrolysate 1 kDa retantate according to the peaks of absorption. After comparing with the standards which contains different molecular weight, these four fractions includes a gel filitrate 1 (GF1) with the molecular weight more than 6512 Da, a gel filitrate 2 (GF2) with the molecular weight between 20806512 Da, a gel filitrate 3 (GF3) with the molecular weight between 1892080 Da and a gel filitrate 4 (GF4) with the molecular weight less than 198 Da.

### Example 12

### Measurement of the Glycerol Release Mediated by Different Gel Fractions Separated by Gel Chromatography

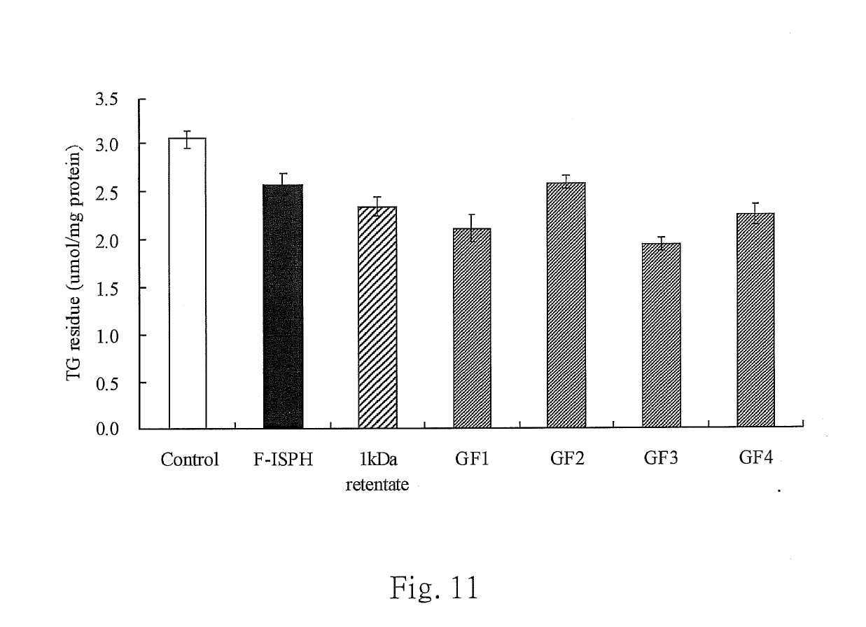

The GF1˜GF4 fractions in example 11, the ISP hydrolysate in example 5 and its 1 kDa retentate are respectively added into the culture medium for 3T3-L1 adipocytes. The flow chart is identical with example 3 to detect the amount of glycerol release in 3T3-L1 adipocytes stimulated by each treatment. The results in this assay are showed in FIG. 11, therein the 3T3-L1 adipocytes cultured without ISP hydrolysate or any separated fractions is the control.

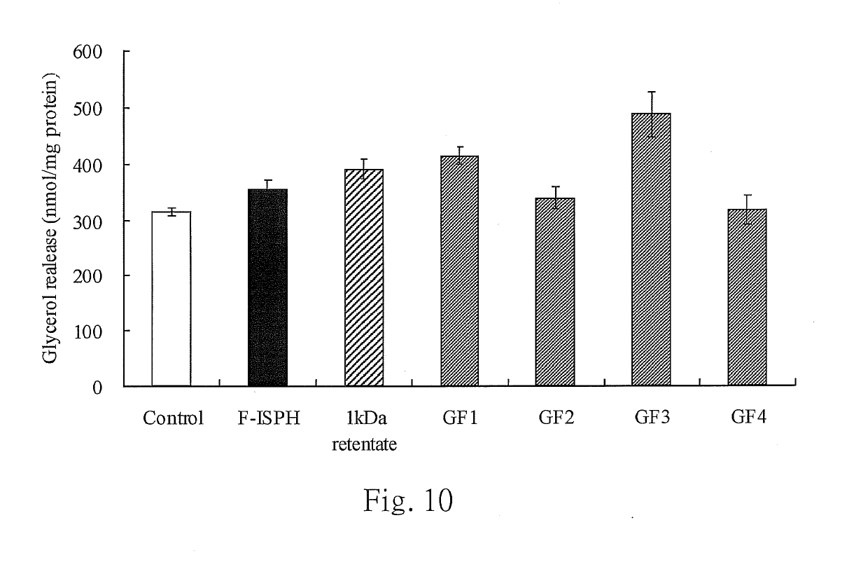

According to the results in FIG. 10, the glycerol release in the 3T3-L1 adipocytes cultured with GF2 and GF4 fractions did not show the obvious difference when compared with control. In contrast, addition of GF1 and GF3 fractions in the cultured medium significantly increases the glycerol release from the basal level, 314.79 nmol/mg protein, in the control group to 415.23 nmol/mg protein and 487.73 nmol/mg protein, respectively. Moreover, the glycerol release in the adipocytes cultured with GF3 fraction reveals significance when compared to the control after the statistical analysis, but the glycerol release in adipocytes cultured with GF1 fraction does not.

### Example 13

### Detection of the Triglyceride Residue in the 3T3-L1 Adipocytes Cultured with Different Fractions Separated by Gel Chromatography

The isolated GF1˜GF4 fractions in example 11, the ISP hydrolysate in example 5 and its 1 kDa retentate are respectively added into the culture medium for 3T3-L1 adipocytes. The 3T3-L1 adipocytes cultured as described method in example 2 are washed with PBS and further cultured to day 11 post-differentiation. The triglyceride residue in 3T3-L1 adipocytes is measured according the detection method in example 7, and result in this measurement is showed in FIG. 11. In triglyceride residue measuring assay, the control experiment is conducted with the culture medium without ISP hydrolysate or any fraction.

FIG. 11 clearly shows that treatment of GF1˜GF4 fractions could significantly reduce the triglyceride residue in the 3T3-L1 adipocytes when compared to the control. Furthermore, the comparison between the triglyceride residue in 3T3-L1 adipocyte with treatment of GF2, GF4 and 1 kDa retentate do not show the obvious difference. In addition, the least triglyceride residue in 3T3-L1 adipocytes is found in the cells treated with GF3 fraction (1.95 μmol/mg protein) which is significantly less than the triglyceride residue in the 3T3-L1 adipocytes treated with GF1 fraction (2.11 μmol/mg protein). Taken together, the results in example 12 and example 13 suggest that the treatment of GF3 fraction in 3T3-L1 adipocytes show 55% increase in glycerol release and 36% reduced triglyceride residue in the adipocytes when compared with control. Therefore, these results indicate that GF3 fraction contains the peptide with best bioactivity for lipolysis-stimulating activity in the cells.

### Example 14

### The Dosage Effects of GF3 Retentate on Glycerol Release in 3T3-L1 Adipocytes

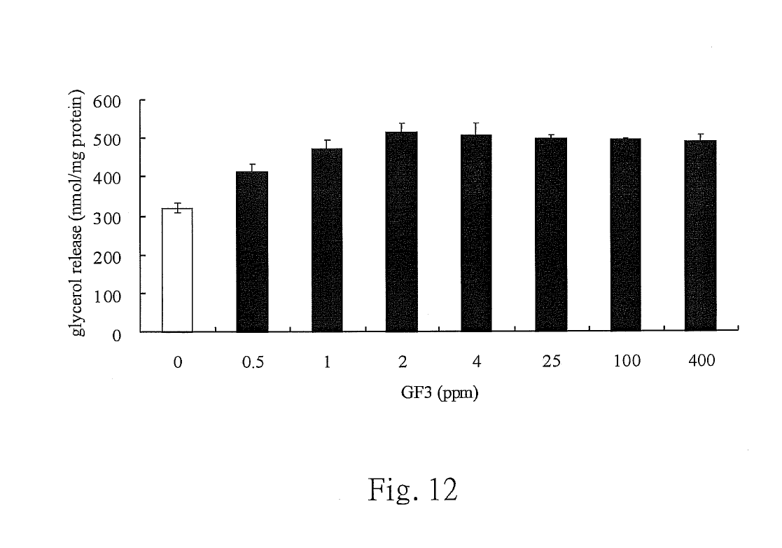

The ISP hydrolysate GF3 fractions are respectively prepared at the concentrations including 0.5, 1, 2, 4, 25, 100 and 400 ppm, and are respectively added in the culture medium for 3T3-L1 adipocytes. 30 μL cultured medium is collected for incubation with glycerol detecting reagent at room-temperature for 5 minutes. The glycerol release in 3T3-L1 adipocytes treated with GF3 fractions in different concentration are measured and calculated from the absorption excited by 520 nm wavelength. The results of glycerol release in the adipocytes with GF3 fractions at different concentrations are showed in FIG. 12 which contains the control experiment without treatment of ISP hydrolysate or any fractions.

The results show that addition of GF3 fractions at any concentration could significantly increase the amount of glycerol release, therein the glycerol release in the cells treated with 1˜400 ppm GF3 fraction are significantly more than that treated with 0.5 ppm GF3 fraction. In addition, addition of GF3 fractions at 2 and 4 ppm show the most glycerol release which increase the glycerol release up to 61% when compared to the control.

### Example 15

### The Dosage Effects of GF3 Retentate on Triglyceride Residue in 3T3-L1 Adipocytes

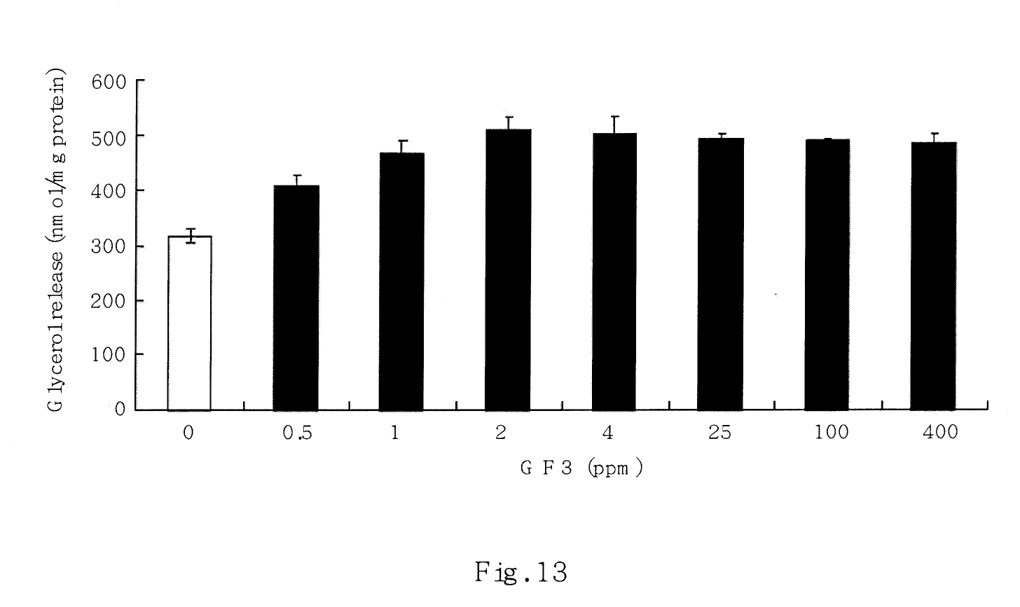

The ISP hydrolysate GF3 fractions at 0.5, 1, 2, 4, 25, 100 and 400 ppm are respectively added into the culture medium for 3T3-L1 adipocytes. The same measuring method in example 7 to detect the triglyceride residue in the adipocytes is performed in this example. The triglyceride residue in the adipocytes is calculated according the absorption excited by 500 nm wavelength, and showed in FIG. 13 which contains control experiment without addition of ISP hydrolysate or any GF3 fraction. With the comparison with control group, triglyceride residue in the adipocytes treated with 0.5400 ppm ISP hydrolysate GF3 fractions are significantly reduced, therein treatments with 2 ppm, 4 ppm and 25 ppm GF3 fractions reveal the least triglyceride residue without any difference from each other.

Taken together, the results in example 14 and example 15 show that addition with more than 0.5 ppm GF3 fractions could significantly increase glycerol release in 3T3-L1 adipocytes. In addition, treatment with 4 ppm GF3 fraction for 3T3-L1 adipocytes shows the most glycerol release and reduces of triglyceride residue in adipocytes from 3.11 umol/mg protein to 1.69 umol/mg protein. Therefore, these results suggest that treatment with 4 ppm GF3 fraction contains the best bioactivity for lipolysis according the statistical analysis.

### Example 16

### Separation of the GF3 Fraction by HPLC

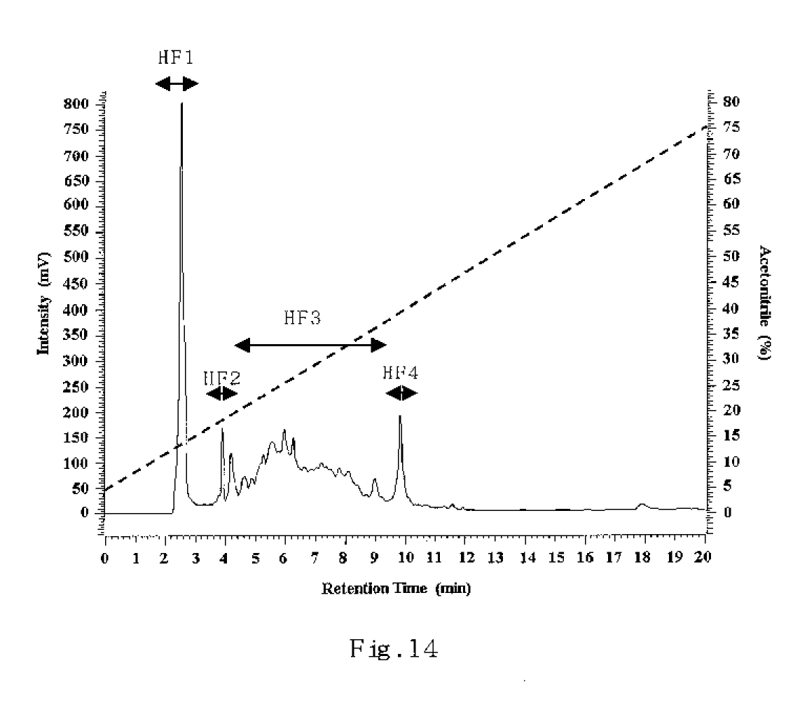

First, the GF3 fraction prepared at a predetermined concentration in example 11 is further separated by using HPLC (HO1100 series) with Develosil™ ODS-HG-5 RPLC column 20 μL of GF3 fraction is injected into the column and followed the separation by mobile phase including ddH2O and acetonitrile. The concentration of acetonitrile is increased from 5% to 75% when the retention time from 0 minute to 20 minutes with the flow rate 1 μL/min for gradient wash to separate the molecules according to their polarity. The result is shown in FIG. 14. Therein, the spectrum analyzed by HPLC contains for fraction including HF1 fraction (HPLC filtrate 1), HF2 fraction (HPLC filtrate 2), HF3 fraction (HPLC filtrate 3) and HF4 fraction (HPLC filtrate 4). Four fractions are collected and freeze-drying for the further analysis.

### Example 17

### Effect of HF1˜HF4 Fractions on Glycerol Release in 3T3-L1 Adipocytes

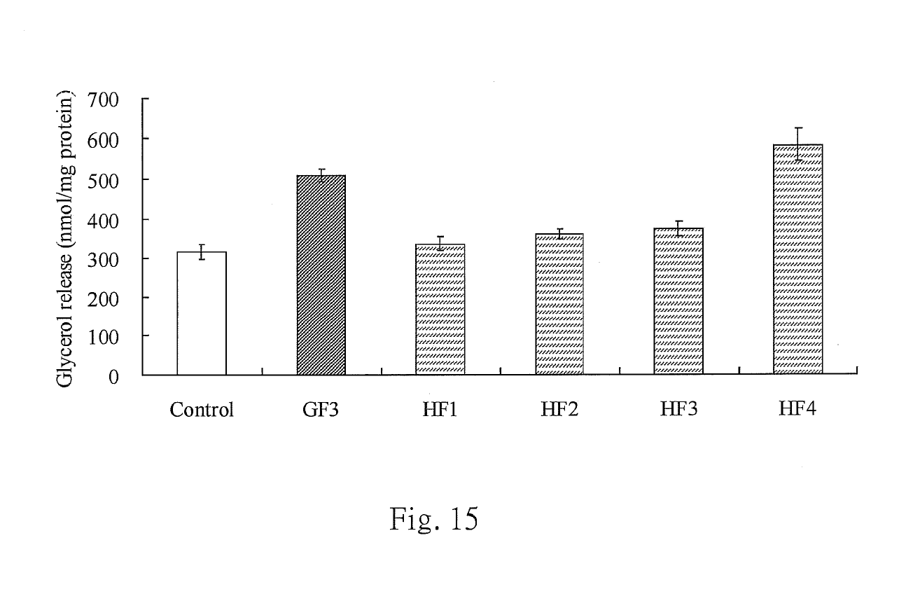

First, the 4 ppm HF1˜HF4 fractions collected in example 16 are respectively added into the culture medium for 3T3-L1 adipocytes until day 11 post-differentiation. In order to measure the glycerol release, the same flow chart described in example 3 is conducted. The amount of glycerol release is calculated according to the absorption excited by 520 nm wavelength after incubation of culture medium and glycerol detecting reagent. The results of glycerol release in adipocytes treated with different fractions are shown in FIG. 15 which contain the control experiment without treatment of ISP hydrolysate or any fractions.

The results in FIG. 15 clearly show that addition of HF2, HF3 and HF4 fractions could significantly increase the glycerol release in adipocytes when compared with control. Especially, addition of HF4 fraction reveals the most obvious effect in increasing glycerol release which is also significantly more than undifferentiated GF3 fraction. Treatment of HF4 fraction for the adipocyte could increase the glycerol release from basal level 317.15 nmol/mg protein to 581.63 nmol/mg protein (increase 83%).

### Example 18

### Effect of HF1˜HF4 Fractions on Triglyceride Residue in 3T3-L1 Adipocytes

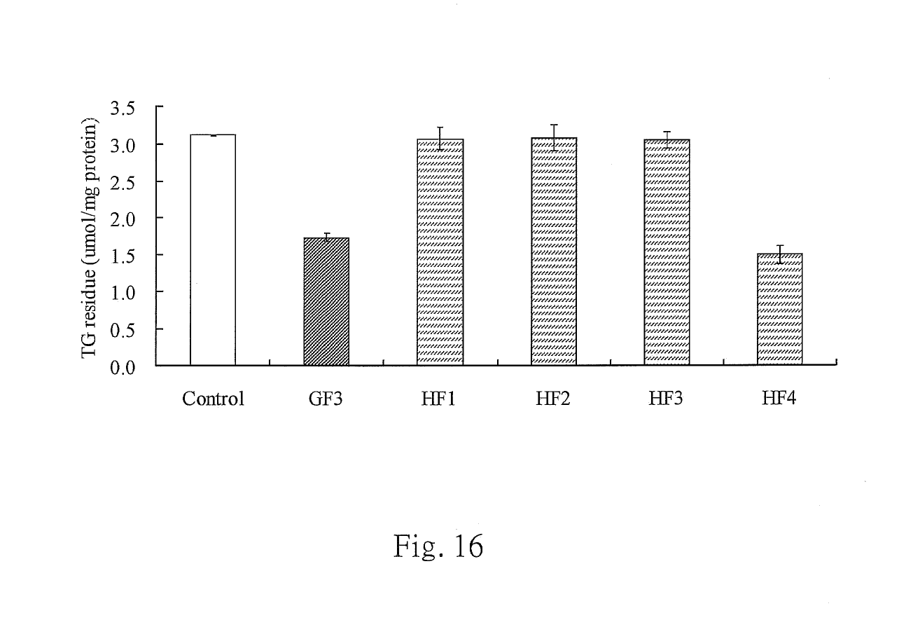

4 ppm HF1˜HF4 fractions prepared in example 16 are respectively added into the culture medium for 3T3-L1 adipocytes. On day 11 post-differentiation, the cell extract in the lysis is collected after centrifugation according to the method described in example 7, and used for measuring triglyceride residue by calculation from the absorption excited by 520 nm wavelength. The results are shown in FIG. 16 which contains the control experiment without treatment of ISP hydrolysate or any fractions.

The results show that treatment of HF4 fraction could significantly reduce 52% of triglyceride residue in adipocytes from the basal level, 3.12 μmol/mg protein, to 1.5 μmol/mg protein when compared with control. In addition, triglyceride residue in the adipocytes treated with HF4 fraction is also significantly less that those treated with GF3 fraction. Taken together, the results in example 17 and example 18 suggest that the isolated HF4 fraction from GF3 fraction by HPLC contains the best bioactivity in lipolysis.

### Example 19

### Separation of HF4 Fraction by HPLC

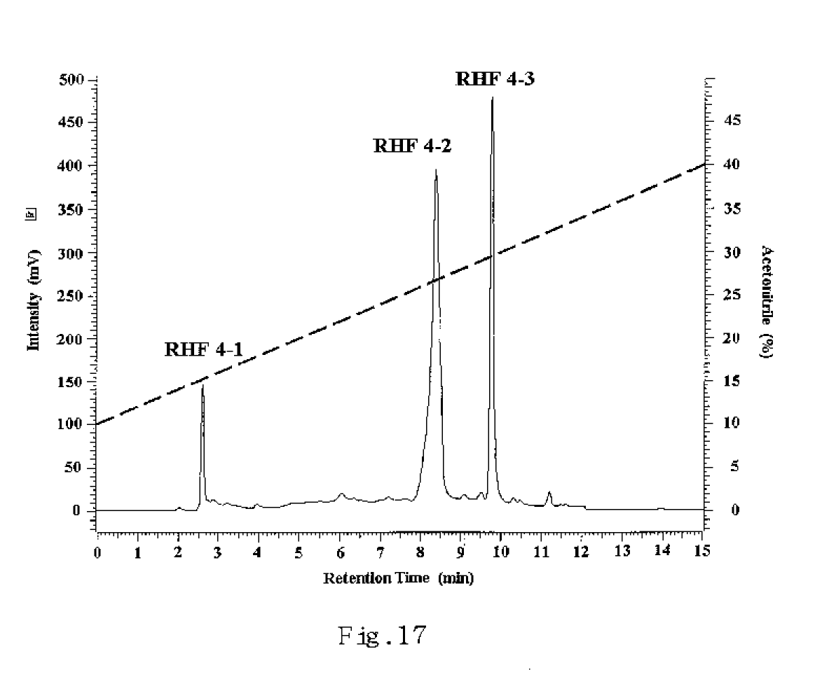

In order to investigate whether the HF4 fraction contain the single peptide which is shown as the hydrophobic peptide in FIG. 15, HF4 fraction is further separated by HPLC with Develosil™ ODS-HG-5 RPLC column. 20 μL of the HF4 fraction is injected into the column which is followed by flow of ddH₂O and acetonitrile as the mobile phase. When the solid phase is washed by the mobile phase, the concentration of acetonitrile is gradually increased from 10% to 40% at 0˜15 minutes with the flow rate at 1.0 mL/minute. The result in FIG. 17 shows that RHF4-1 fraction (repeat HF4-1), RHF4-2 fraction (repeat HF4-2) and RHF4-3 fraction (repeat HF4-3) are isolated according to the histogram analyzed by HPLC.

### Example 20

### Effect of RHF4-1-RHF4-3 Fractions on Glycerol Release in 3T3-L1 Adipocytes

First, 4 ppm RHF4-1˜RHF4-3 fractions isolated according the example 19 are respectively added into the culture medium for 3T3-L1 adipocytes for the further culture. On day 11 post-differentiation, the glycerol release in the medium from adipocytes is measured according to the method in example 3. The amount of glycerol release in the culture medium is calculated by the absorption excited by 520 nm wavelength.

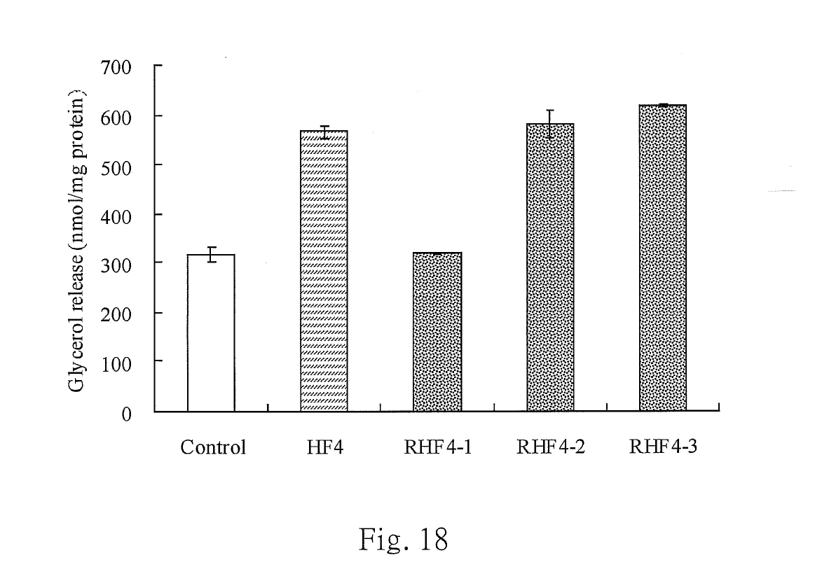

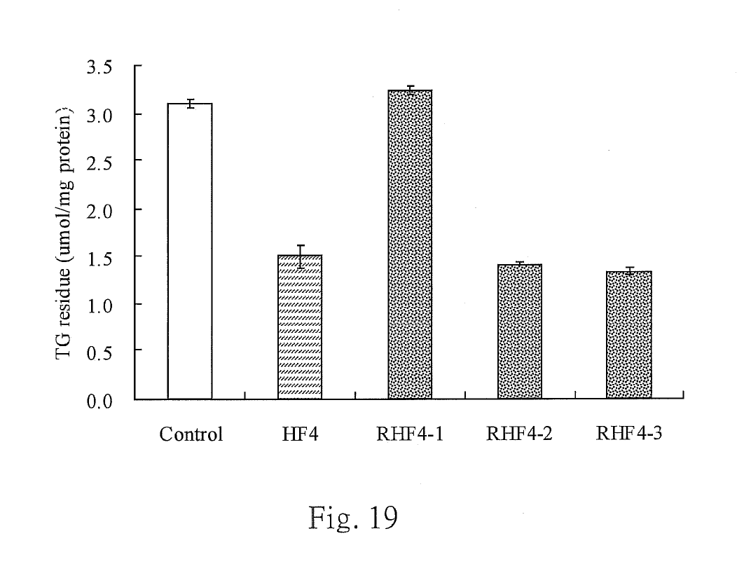

The data are showed in FIG. 18 which contain the control experiment without treatment of ISP hydrolysate or any isolated fractions. The FIG. 19 reveals that addition of RHF4-1 or RHF4-3 fractions significantly increase the glycerol release from basal level up to 580.59 nmol/mg protein (increase 84%) and 615.87 nmol/mg protein (increase 95%), respectively.

### Example 21

### Effect of RHF4-1˜RHF4-3 Fractions on Triglyceride Residue in 3T3-L1 Adipocytes

4 ppm RHF4-1˜RHF4-3 fractions collected in example 19 are respectively added into the culture medium for 3T3-L1 adipocyte. On day 11 post-differentiation, the cell extract collected according to example 7 is used for determinating triglyceride residue by calculation according to the absorption excitated by 520 nm wavelength. The results are showed in FIG. 19 which contains the control experiment without treatment of ISP hydrolysate or any fractions.

The result reveals that addition of RHF4-2 and RHF4-3 fractions could significantly reduce the triglyceride residue from 3.1 μmol/mg protein in control to 1.42 μmol/mg protein (decrease 54%) and 1.34 μmol/mg protein (decrease 57%), respectively.

Taken together, the results in example 20 and example 21 reveal that the peptides in RHF4-2 and RHF4-3 fractions possess the best bioactivity for lipolysis.

### Example 22

### Determination of the Amino Acid Sequence of Peptides in RHF4-2 and RHF4-3 Fractions

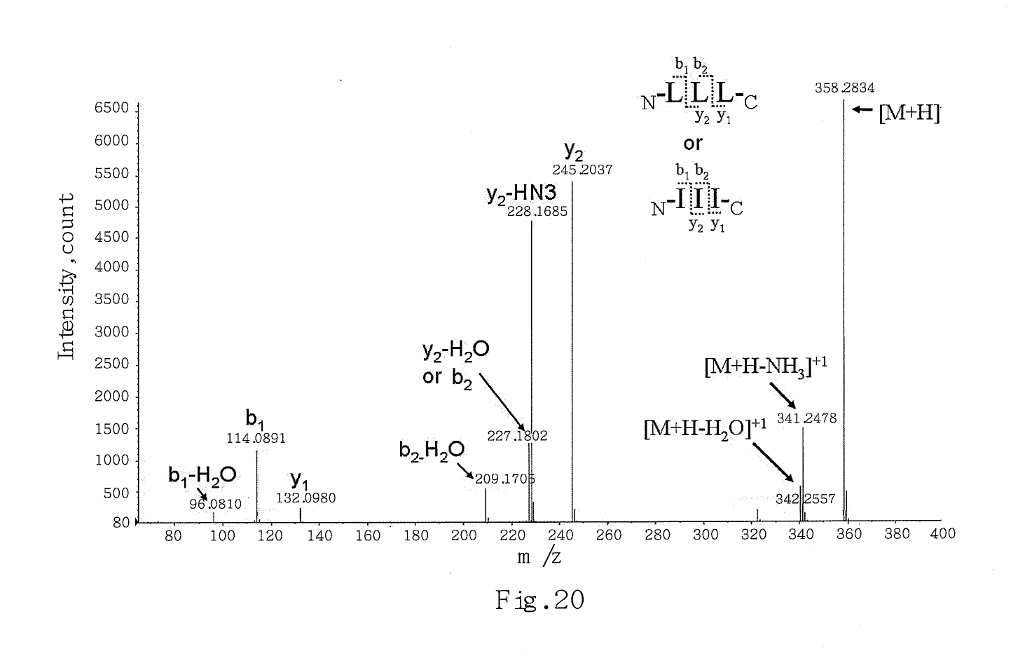

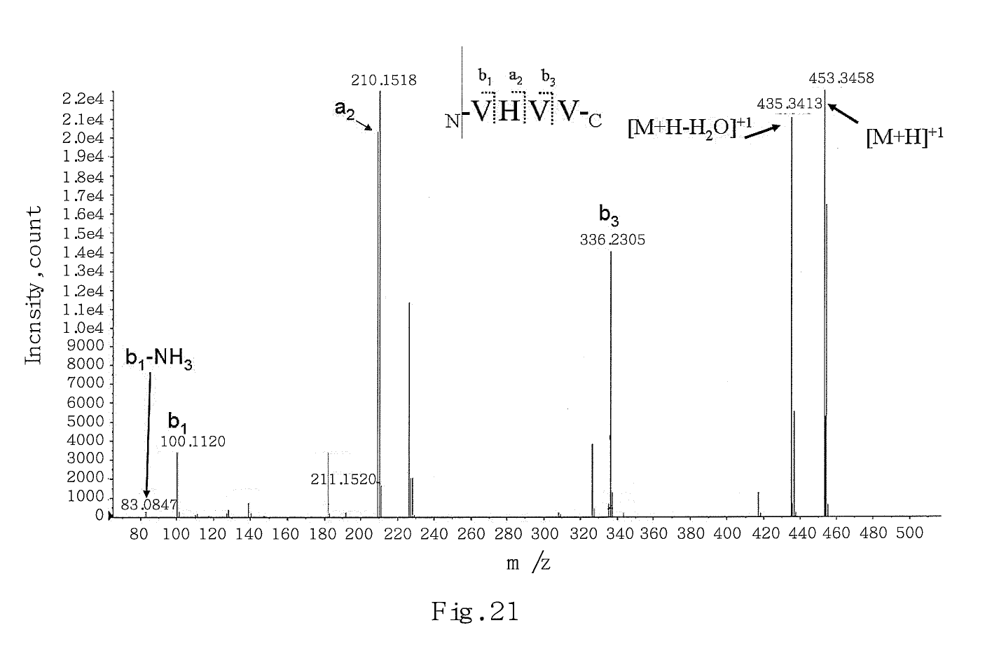

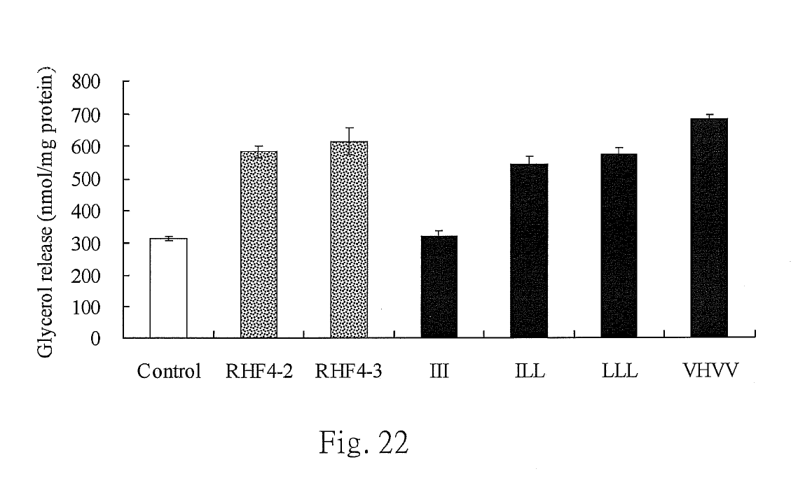

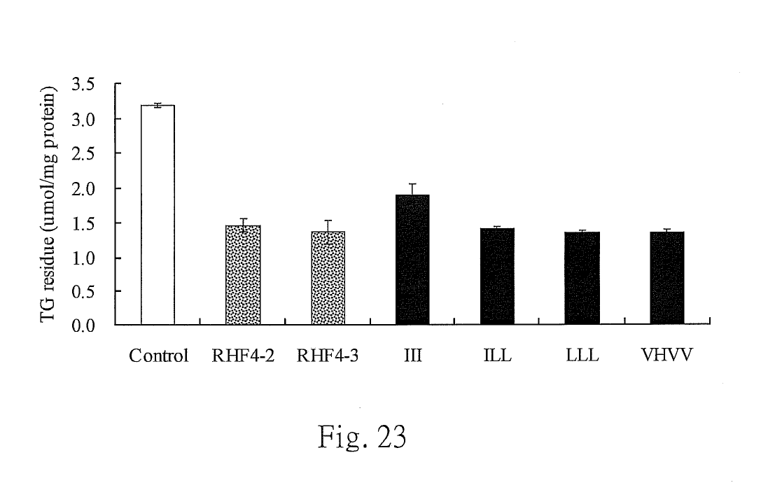

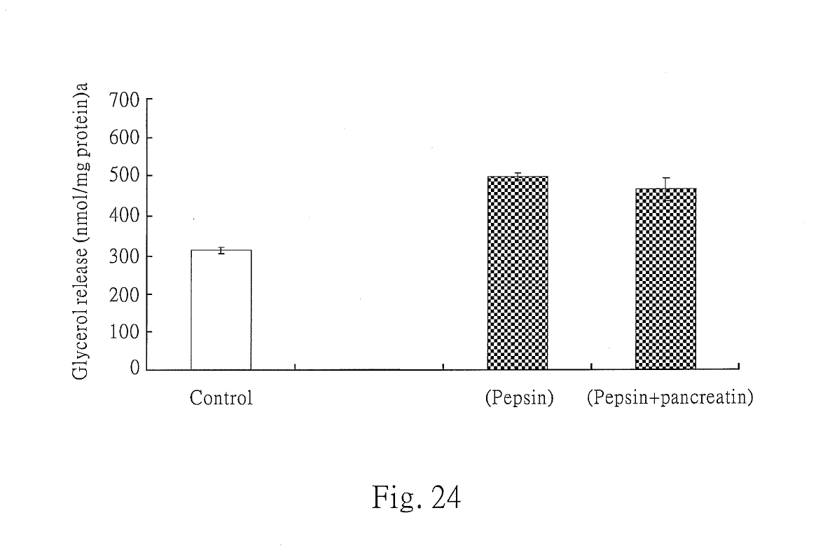

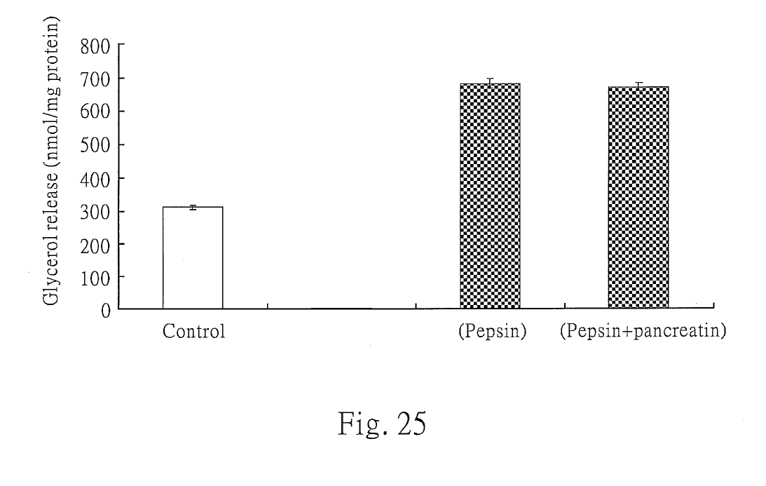

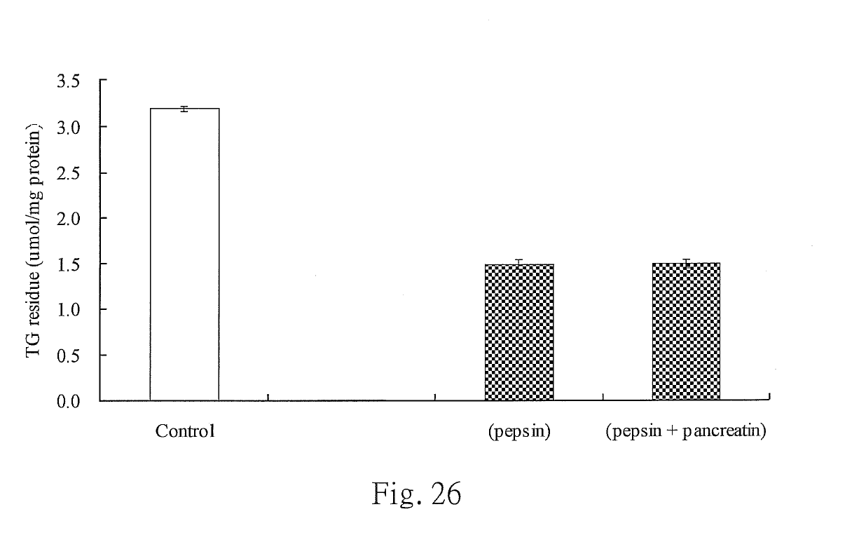

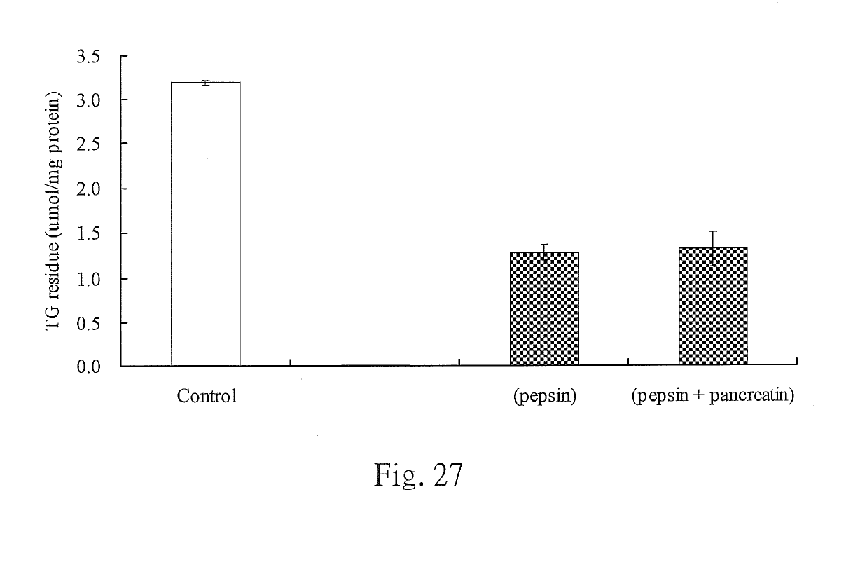

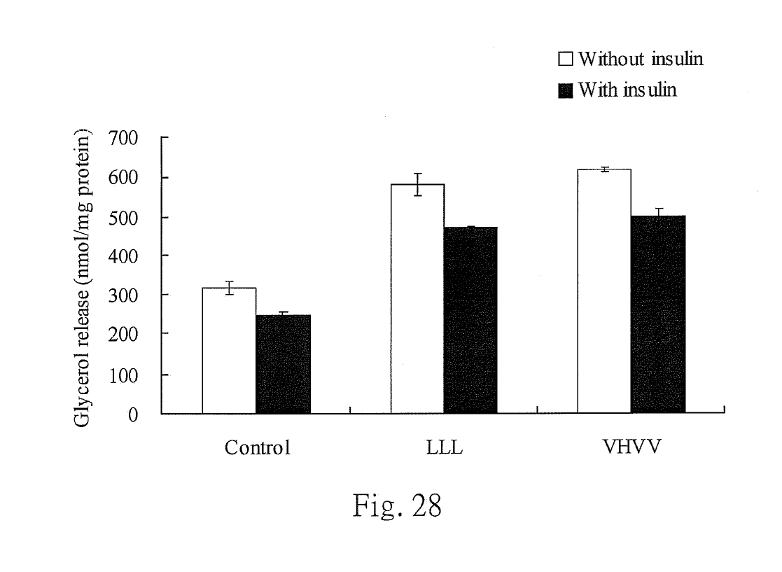

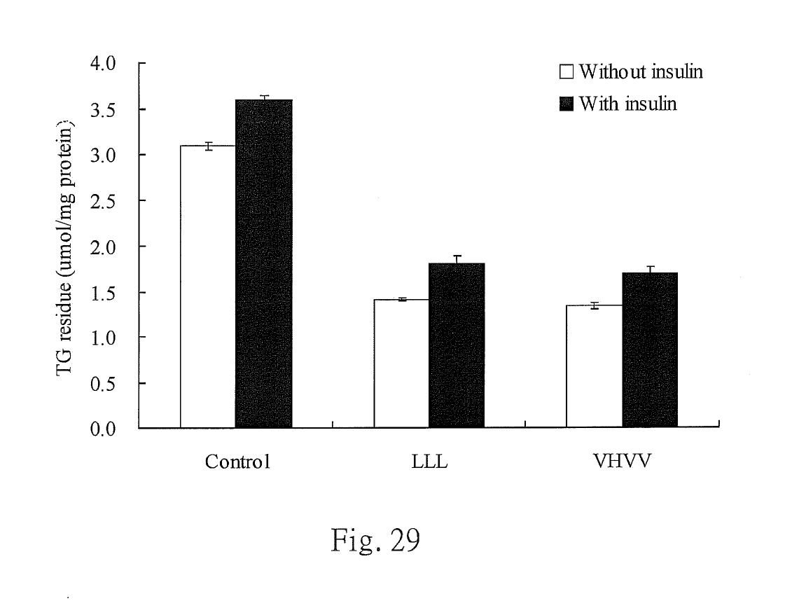

The RHF4-2 and RHF4-3 fractions are further respectively analyzed by mass spectrometer using LC/MS/MS. The result of the finger print of LC/MS/MS is compared with the database and shown in FIGS. 20 and 21, wherein the FIG. 20 is the mass spectrum of RHF4-2 fraction and the FIG. 21 is the mass spectrum of RHF4-3 fraction.

FIG. 20 suggests that RHF4-2 fraction contains the tripeptide composed of leucine (Leu) and isoleucine (Ile). Therefore, the possible amino acid sequence combinations of the tripeptide in RHF4-2 fraction includes Leu-Leu-Leu, Leu-Leu-Ile, Leu-Ile-Leu, Leu-Ile-Ile, Ile-Leu-Leu, Ile-Leu-Ile, Ile-Ile-Leu and Ile-Ile-Ile.

Moreover, FIG. 21 suggests that the RHF4-3 fraction contains the tetrapeptide which is composed of Val-His-Val-Val.

### Example 23

### Effect of the Chemical Synthetical Peptides in Lipolysis

In this example, the synthetical peptides including Ile-Ile-Ile (III), Ile-Leu-Leu (ILL), Leu-Leu-Leu (LLL) and Val-His-Val-Val (VHVV) are used to determine the bioactivity in lipolysis in the culture adipocyte.

4 ppm RHF4-2, RHF4-3 fractions isolated in example 19 and the above-identified four synthetical peptides are respectively added into the culture medium for 3T3-L1 adipocytes. The glycerol release and triglyceride residue in the adipocytes are measured by calculation of the absorption excited by 520 and 500 nm wavelength, respectively. The results of glycerol release and triglyceride residue in the adipocytes with these treatments are shown in FIGS. 22 and 23 which contain the control experiment without treatment of ISP hydrolysate or any fraction. FIG. 22 reveals the effect of RHF4-2, RHF4-3 fractions and the synthetical peptides on glycerol release in adipocytes. Moreover, FIG. 23 shows the effect of RHF4-2, RHF4-3 fractions and the synthetical peptides on triglyceride residue in adipocytes.

The FIG. 22 reveals that addition of RHF4-2 fraction, Ile-Leu-Leu and Leu-Leu-Leu synthetical peptides significantly increase the glycerol release in 3T3-L1 adipocytes from the basal level, 312.3 nmol/mg protein, in control up to 581.61 nmol/mg protein, 540.81 nmol/mg protein and 571.2 nmol/mg protein. In addition, treatments of RHF4-3 fraction and Val-His-Val-Val synthetical peptide also obviously increase the glycerol release in adipocytes from basal level up to 614.4 nmol/mg protein and 682.91 nmol/mg protein, respectively.

Therefore, the RHF4-2 fraction, RHF4-3 fraction or the synthetical peptides including Ile-Leu-Leu, Leu-Leu-Leu and Val-His-Val-Val could increase the glycerol release in adipocyte.

Furthermore, FIG. 23 reveals that addition of RHF4-2 fraction, Ile-Ile-Ile, Ile-Leu-Leu and Leu-Leu-Leu synthetical peptides significantly reduce the triglyceride residue in 3T3-L1 adipocytes with the comparison of control. In this assay, addition of RHF4-2 fraction, Ile-Leu-Leu and Leu-Leu-Leu synthetical peptides reduce the triglyceride residue from 3.2 μmol/mg protein in control to 1.46 μmol/mg protein, 1.41 μmol/mg protein and 1.34 μmol/mg protein. In addition, both treatment of RHF4-3 fraction and the synthetical peptides Val-His-Val-Val significantly reduce the triglyceride residue in the adipocytes from 3.2 μmol/mg protein to 1.36 μmol/mg protein.

Collectively, the results in this example suggest that the synthetical peptides Ile-Ile-Ile possesses the bioactivity in anti-lipogenesis, and the synthetical peptides Ile-Leu-Leu, Leu-Leu-Leu, Val-His-Val-Val reveal the bioactivity in stimulating lipolysis.

### Example 24

### Effect of the Resistance of the Synthetical Peptides in the Mimic Gastrointestinal Environment

Prepared 1% synthetical peptides Leu-Leu-Leu and Val-His-Val-Val are respectively incubated in 0.1M KCl/HCl buffer (pH value 2.0) for reaction at 37° C. in the reactor. Then adding pepsin with substrate-enzyme ratio at 25:1 and incubating for 4 hours is performed to mimic the environment in stomach. Following the incubation, the solution is neutralized by addition of 2N NaOH and heating some aliquot for 15 minutes to terminate the pepsin activity for freeze storage. The remainder is further incubated with pancreatin with substrate-enzyme ratio at 25:1 for 4 hours to mimic the intestinal environment. After termination of the enzyme activity by heating in boiled water and followed by cooling, the sample is freeze stored for the further analysis.

After temperature returned, the each frozen sample processed in the mimic gastrointestinal environment is centrifuged at 10,000×g for 40 minutes. After centrifugation, the supernatant is collected and filtrated through 0.22 μm filter, and added into the culture medium for 3T3-L1 adipocytes. On day 11 post-differentiation, the supernatant or cell extract are collected as the methods described in example 3 and example 7 for measuring the glycerol release and triglyceride residue in the adipocytes. The glycerol release and triglyceride residue in the adipocytes are calculated from the absorption excited by 500 and 520 nm wavelength. The results are showed in FIGS. 24 to 27 which contain the control experiment without adding synthetical peptides.

In FIGS. 24 to 27, both synthetical peptides Leu-Leu-Leu and Val-His-Val-Val treated with the gastrointestinal enzymes are able to significantly increase the glycerol release and decrease triglyceride residue in the adipocytes. Therefore, the bioactivity for lipolysis of the synthetical peptides Leu-Leu-Leu and Val-His-Val-Val are resistant to enzyme activity of gastrointestinal enzymes.

### Example 25

### Effect of the Synthetical Peptides in the Presence of Insulin

The adipocytes respectively cultured with the synthetical peptides Leu-Leu-Leu and Val-His-Val-Val are further respectively treated with insulin. As the preparing methods described in example 3 and example 7, the glycerol release and triglyceride residue in adipocyte are measured according to the absorption excited with 520 and 500 nm wavelength. The results of the insulin effect are showed in FIG. 28 and FIG. 29 which contain control experiment with insulin but not synthetical peptides.

The results show that the synthetical peptides Leu-Leu-Leu and Val-His-Val-Val significantly increase glycerol release and decrease triglyceride residue in the adipocytes under insulin treatment when compared with control. Therefore, the synthetical peptides Leu-Leu-Leu and Val-His-Val-Val still possess bioactivity for lipolysis within the environment containing insulin.

Collectively, all examples described above suggest that this invention provides the optimal hydrolysis condition to obtain the ISP hydrolysate for glycerol release in adipocytes through stimulating phosphorylation of HSL. Furthermore, the single peptide isolated from ISP hydrolysate by HPLC also significantly increases the glycerol release and reduces triglyceride residue in the adipocytes. Finally, the amino acid sequence is further determined by LC/MS/MS. In addition, the synthetical peptides which possess the bioactivity for lipolysis in adipocytes are resistant to the digestion of gastrointestinal enzymes and effect of insulin. Therefore, this invention claims that the single peptide isolated from ISP could be applied in medical utilization or the related healthy foods for reducing body weight. It is helpful for our health through more efficiently reducing the incidence of obesity.

The above-mentioned specification is only for detailedly describing the examples of the invention and shall not be construed as a limitation of the scope of the invention Thus, any modification or change without departing from the characteristics of the invention or any equivalent thereof shall be included in the scope of the invention defined in the following claims.

What is claimed is:

1. A method for preparing a lipolysis-stimulating soy protein hydrolysate, proceeding a hydrolysis reaction, which is a predetermined concentration of soy protein mediated by Flavourzyme in a predetermined hydrolysis condition, to obtain a soy protein hydrolysate, wherein:
the hydrolysis condition being at pH value 7˜7.5, at reaction temperature 40˜50° C. and at hydrolysis time 100˜150 minutes.

2. The method for preparing a lipolysis-stimulating soy protein hydrolysate of claim 1, wherein the soy protein selected from groups having isolated soy protein, defatted soy flour and soy protein concentrate.

3. The method for preparing a lipolysis-stimulating soy protein hydrolysate of claim 1, wherein the Flavourzyme versus soy protein is 1:100.

4. The method for preparing a lipolysis-stimulating soy protein hydrolysate of claim 2, wherein the Flavourzyme versus soy protein is 1:100.

5. The method for preparing a lipolysis-stimulating soy protein hydrolysate of claim 1, wherein the pH values is 7.12.

6. The method for preparing a lipolysis-stimulating soy protein hydrolysate of claim 2, wherein the pH values is 7.12.

7. The method for preparing a lipolysis-stimulating soy protein hydrolysate of claim 1, wherein the reaction temperature is 48.8° C.

8. The method for preparing a lipolysis-stimulating soy protein hydrolysate of claim 2, wherein the reaction temperature is 48.8° C.

9. The method for preparing a lipolysis-stimulating soy protein hydrolysate of claim 1, wherein the hydrolysis time is 124.9 minutes.

10. The method for preparing a lipolysis-stimulating soy protein hydrolysate of claim 2, wherein the hydrolysis time is 124.9 minutes.

11. A isolated functional peptide, has an amino acid sequence being Val-His-Val-Val.

12. The isolated functional peptide of claim 11, being used for increasing the glycerol release in adipocytes of an organism.

13. The isolated functional peptide of claim 11, being obtained from a hydrolysis of a soy protein is mediated by Flavourzyme.

14. The isolated functional peptide of claim 12, being obtained from a hydrolysis of a soy protein is mediated by Flavourzyme.

15. The isolated functional peptide of claim 13, wherein the soy protein selected from groups having isolated soy protein, defatted soy flour and soy protein concentrate.

16. The isolated functional peptide of claim 14, wherein the soy protein selected from groups having isolated soy protein, defatted soy flour and soy protein concentrate.

17. An isolated functional peptide, which is composed of three amino acids, wherein;
the first amino acid selected from Leu and Ile;
the second amino acid selected from Leu and Ile;
the third amino acid selected from Leu and Ile.

18. The isolated functional peptide of claim 17, being used for increasing the glycerol release in adipocytes of an organism.

19. The isolated functional peptide of claim 17, being obtained from a hydrolysis of a soy protein is mediated by Flavourzyme.

20. The isolated functional peptide of claim 18, being obtained from a hydrolysis of a soy protein is mediated by Flavourzyme.

21. The isolated functional peptide of claim 19, wherein the soy protein is selected from groups having isolated soy protein, defatted soy flour and soy protein concentrate.

22. The isolated functional peptide of claim 20, wherein the soy protein is selected from groups having isolated soy protein, defatted soy flour and soy protein concentrate.

23. A medical compound of reducing weight, having an effective component being a functional peptide, wherein the peptide has an amino acid sequence selected from the following (1) and (2):
(1) Val-His-Val-Val;
(2) the peptide composed of three amino acids, wherein;
the first amino acid selected from Leu and Ile;
the second amino acid selected from Leu and Ile;
the third amino acid selected from Leu and Ile.

24. The medical compound of claim 23, wherein the functional peptide is in a soy protein hydrolysate.
